(19) United States
(12) Patent Application Publication
(10) Pub. No.: US 2025/0041353 A1
(43) Pub. Date: Feb. 6, 2025

(54) USE OF CD90+ HUMAN AMNIOTIC EPITHELIAL CELLS IN TREATMENT OF GRAFT VERSUS HOST DISEASE

(71) Applicants: SHANGHAI SAIAO BIOTECHNOLOGY CO., LTD, Shanghai (CN); PEKING UNIVERSITY PEOPLE'S HOSPITAL, Beijing (CN); ZHEJIANG UNIVERSITY, Hangzhou (CN)

(72) Inventors: Xiaojun HUANG, Beijing (CN); Luyang YU, Hangzhou (CN); Chuanyu ZHANG, Shanghai (CN); Pengjie YANG, Hangzhou (CN); Xiangyu ZHAO, Beijing (CN); Yaohui KOU, Hangzhou (CN); Weixin YUAN, Shanghai (CN); Jinying LI, Hangzhou (CN); Jia LIU, Hangzhou (CN)

(21) Appl. No.: 18/696,539

(22) PCT Filed: Sep. 30, 2021
(86) PCT No.: PCT/CN2021/122208

§ 371 Date: Mar. 28, 2024

(51) Int. Cl. A61K 35/50; A61P 37/06; C12M 1/00; C12N 5/073

(52) U.S. Cl. CPC A61K 35/50; A61P 37/06; C12M 47/04; C12N 5/0605; C12N 2501/11; C12N 2501/115; C12N 2501/998

(57) ABSTRACT

A method for treating a subject suffering from a graft versus host disease (GvHD) or with the risk of GvHD, comprising: selecting a CD90⁺hAEC cell population with a stronger immunoregulation capability as a primary therapeutic cell. Research on related action mechanisms finds that the stem cell pluripotent markers SSEA4, OCT4 and NANOG of CD90⁺hAECs are significantly higher than general hAECs, and have better immunomodulatory functions, and therefore, the CD90⁺hAECs cells are used as therapeutic means to obtain better clinical results.

### TECHNICAL BACKGROUND

Hematopoietie stem cell transplantation (HSCT) was first introduced into clinical practice by American scientist Professor Thomas in the 1950s, and after decades of development, HSCT has been used for the treatment of a large number of malignant hematological disorders and metabolic inborn defects, etc. It is the only existing means of eradicating hematologic tumors.

In hematopoietic transplantation, graft-versus-leukaemia (GVL) or graft-versus-tumor (GVT) is the main mechanism for cancer treatment. In contrast, graft-versus-host disease (GVHD), a major complication of HSCT, is the leading cause of post-transplant mortality. According to statistics, about 50% of patients who receive hematopoietic transplantation will develop symptoms of GVHD, thus severely limiting the widespread implementation of this important therapy.

GVHD is a type of immune disorder that affects multiple organ systems, including the gastrointestinal tract, liver, skin, kidneys and lungs, which seriously threatens patients' quality of life and survival time after transplantation. Based on the duration of clinical symptoms, GVHD can be divided into two categories: acute GVHD (aGVHD) and chronic GVHD (cGVHD).

GVHD is caused by an immune response resulting from the response of donor T-cells to genetically determined proteins (mainly HLA human leukocyte antigens, histocompatibility antigens) on the recipient's cells, and therefore the frequency of prevalent aGVHD is directly related to the level of HLA mismatch. The ideal donor and recipient are HLA-A/B/C/DRB1 fully matched.

The pathogenesis of GVHD can be summarized as three processes: first, the activation of antigen-presenting cells (APCs, antigen-presenting cells); second, the activation, proliferation, differentiation and migration of donor T cells cause an inflammatory storm; third, the damage of target organs.

From animal experiments, T cells play a central role in the pathogenesis of GVHD. There are three strategies for removing T cells: removing T cells in vitro followed by transplantation; sorting CD34+ stem cells in vitro; and specifically removing T cells in vivo by using antibodies. These strategies have shown good efficacy against GVHD (regardless of aGVHD or cGVHD). However, unfortunately these treatment options come at the cost of high transplant failure rate, post-transplant infection, and high recurrence rate. In addition, other main methods to prevent and treat GVHD are to perform non-targeted immunosuppressive treatment on the recipient, such as cyclosporin-A (CsA), mycophenolate mofetil, MMF), steroids, etc., but these regimens also weaken the GVL effect, and the results show that the probability of cancer recurrence increases.

In recent years, it has been reported that cell therapy programs such as T regulatory cells (Treg), regulatory γδT cells (γδTreg), and mesenchymal stem cells (MSC) have been used in experimental animals, showing certain curative effects. However, in clinical trials, MSCs have been reported to promote the risk of tumor recurrence. Treg cannot be promoted in clinical application due to its limited source and difficulty in in vitro expansion.

CD4+ T cells have been widely reported that T cells are closely related to GVHD, and the balance among the four main subgroups Th1, Th2, Th17 and Treg has a great influence on the incidence and severity of GVHD. In acute GVHD as an inflammatory process, CD4+ polarization toward Th1 was detected. In recent years, studies have discovered a new type of CD4+ cell subset that can produce IL-17, namely Th17. Subsequent mouse disease models and clinical studies found that it plays an important role in the initiation of inflammation and tissue damage in acute GVHD. Studies have shown that Th2 is involved in GVHD and is related to lung damage and chronic GVHD. Among the four main subgroups of CD4+, only Treg has been identified and found to be associated with GVHD. It is widely believed that it can inhibit and improve GVHD while still retaining the efficacy of GVL. In summary, the research on the polarization remodeling of CD4+ subset is particularly important.

Human amniotic epithelial cells (hAECs) are cells isolated from the amniotic membrane on the side of the placenta closest to the fetus. Placenta is a waste product of pregnant women after childbirth, so there are no ethical issues with cells derived from it. Anatomically, the placenta can be divided into three main layers of tissue structure from the inside to the outside: amniotic epithelium, chorion, and uterine decidua, and the origin of each layer of tissue is completely different. The decidua is of maternal origin, the chorion is of trophoblast origin, and the amniotic epithelium comes from the epiblast eight days after fertilization, which is the same as embryonic stem cells (ESCs). hAECs are derived from the embryonic inner cell mass. Miki et al. demonstrated that hAECs can express some of the characteristic makers of pluripotent stem cells (e.g., ESCs), such as Oct4, Sox2, Nanog, SSEA-3, SSEA-4, etc., indicating that hAECs may have the potential to differentiate into the three germ layer tissues similar to that of ESCs. This inference was confirmed by in vitro differentiation experiments. However, unlike ESCs, hAECs were negative for teratoma in vivo, mainly due to their lack of telomerase activity. Therefore, hAECs have no risk of tumorigenesis (including benign tumors, sarcomas, and carcinomas) when used as cell therapy.

In addition, hAECs hardly express MHC type II molecules on the cell surface, so they will not cause inflammation, allergies, and immune reactions, and the requirements for transplant matching are also reduced accordingly. The separation process of hAECs is relatively simple. Except for a small amount of blood cell clumps, there is almost no other type of cell contamination on the amniotic membrane after scraping and washing, and blood cells are suspension cells when they are not stimulated, so it can be removed by cell culture and then fluid exchange. According to our experimental statistics and those of foreign scientists, there are approximately 80 to 300 million hAECs per human amniotic membrane. Under the condition of the presence of EGF, hAECs have strong proliferative ability, which can proliferate for one generation in about 36 hours, and can maintain vigorous proliferation in the previous generation (within about 10 generations). These advantages ensure that we can obtain sufficient quantities of high-purity cell products to meet the requirements of clinical treatment.

In addition to stemness, another important property of hAECs is immunoregulation. As early as the beginning of the 20th century, researchers began to try to use amniotic membrane as a graft material to repair patients' skin damage, and achieved good results. Further research found that amniotic membrane has the effect of anti-rejection and inhibiting the growth of wound bacteria, suggesting that amniotic membrane may have some kind of immune regulation. Ueta et al. found that amniotic membrane has the function of inhibiting mixed lymphocyte reaction. Li et al. further found that the main functional cells of the amniotic membrane that exerted the immunomodulatory effect were hAECs, which, through the secretion of immune-suppressing factors, inhibited the chemotaxis of neutrophils and macrophages, and inhibit the proliferation of T and B cells stimulated by mitogens. Other scientists have reported that amniotic membrane also induces apoptosis in IFNγ-activated macrophages. Interestingly, through a series of in vitro experiments, amniotic membrane was found to inhibit the proliferation of a variety of solid tumor cell lines. Kang et al. found that hAECs inhibited the proliferation of breast cancer cell lines in vitro, and in vivo inhibited the enlargement of breast cancer tumors in nude mice and prolonged the survival time of mice. With respect to tumor suppression, it is now mainly believed that hAECs act by both inhibiting tumor angiogenesis and promoting apoptosis of tumor cells.

The present inventor further study and find that among the hAECs obtained according to the above isolation and culture method, there is a group of CD90⁺ hAECs whose expression of stem cell pluripotency markers SSEA4, OCT4 and NANOG is significantly higher than that of other CD90⁻hAECs, and has better immunomodulatory function, so this cell is used as a therapeutic method to get better clinical effect.

### SUMMARY

The present invention provides a method of treating a subject suffering from graft-versus-host disease (GvHD) or at risk of suffering from graft-versus-host disease, generally comprising administering to the subject human amniotic membrane epithelial cells (hAECs) that are effective to ameliorate at least one symptom or clinical sign of the graft-versus-host disease as compared to a suitable control subject. In order to further improve the clinical treatment effect, a cell subset of CD90⁺ hAECs with potent immunomodulatory capabilities is selected as the primary therapeutic cells.

In another embodiment of the invention, the present invention relates to the use of human amniotic epithelial cells or cell preparations thereof in the preparation of drugs for treating and/or improving graft-versus-host disease, wherein said human amniotic epithelial cells (hAECs) is CD90⁺ hAECs cell subset.

In another embodiment of the invention, the present invention relates to the use of effective doses of CD90⁺ human amniotic epithelial cells or their cell preparations that can be used alone or in combination with other drugs to treat and/or improve graft-versus-host disease.

In another embodiment of the invention, the cell preparation comprises human amniotic epithelial cells and a pharmaceutically acceptable carrier.

In another embodiment of the invention, the present invention relates to a method for isolating amniotic epithelial cells from amniotic tissue, comprising the following steps:
(1) Obtain amniotic membrane from placental tissue through mechanical separation;
(2) The amniotic membrane is washed and then digested with digestive enzymes, and the digested liquid is centrifuged to obtain human amniotic membrane epithelial cells;
(3) Sort out CD90⁺ human amniotic epithelial cells (hAECs).

The present invention investigates the potential ability of CD90+ human amniotic epithelial cells in the treatment of graft-versus-host disease and explores its therapeutic mechanism. The results show that the CD90⁺ hAECs and PBMC co-transplanted treatment group and the unscreened hAECs treatment group have a better inhibitory effect on aGVHD, significantly improves the clinical and pathological phenotypes of the mice compared to the disease group, as well as significantly improves the survival rate of the mice. Using the animal model of the present invention, it is demonstrated that the method can effectively reduce the infiltration of inflammatory cells into target organs caused by aGVHD, as well as significantly reduce target organ lesions. hAECs are also found to have a pro-apoptotic effect on a number of leukemia cell lines, and can be used for the treatment of graft-versus-host disease, which will have broad prospects in clinical application for the treatment of graft-versus-host disease.

### DESCRIPTION OF DRAWINGS

FIG. 1. The proportion of CD3 and CD45 in each group. CD3 is a type of antigen on the surface of T lymphocytes, CD3+ is mature T lymphocytes; CD45 molecules are expressed on all leukocytes, known as leukocyte common antigens. FACS results showed that after 2 weeks of PBMC implantation, human CD3 and human CD45 molecules were expressed on all leukocytes in the aGVHD model mice, and the chimerism rate was more than 50%. The implantation of both CD90+hAECs and unscreened hAECs did not significantly affect PBMC implantation. Furthermore, we found that almost all implanted CD45+ cells co-expressed CD3. The humanized aGVHD model was successfully constructed and was not affected by CD90+hAECs.

FIG. 2 Isolation of CD90+ hAECs with their immunomodulatory properties. Flow cytometric detection of CD29, CD166, and CD 90 in isolated hAECs. Immunomodulatory properties of hAEC and functional characterization of the CD90+ population. Unsorted hAECs or sorted CD90+ and CD90-hAECs were cocultured with PHA-activated PBMCs for 3 days. TNFα, IL2, IL17 and IL10 in the culture medium were detected by ELISA. (A) Representative flow cytometry gating for CD90⁺ and CD90⁻ hAEC sorting. (B-E) ELISA detection of cytokines TNFα, IL10, IL17, and IL2 in hAECs culture medium.

FIG. 3 Illustration of the appearance of mice in each group. Two weeks after PBMC implantation, mice implanted with PBMC alone began to gradually develop a series of typical clinical symptoms of aGVHD: weight loss, decreased activity, lethargy, severe hunched back, hair loss, diarrhea, etc. Both the unselected hAECs treatment group and the CD90⁺hAECs treatment group improved the clinical symptoms of aGVHD mice.

FIG. 4 Graphical representation of changes in body weight of mice in each group over time. hAECs implantation not only improved the quality of survival of diseased mice, but more importantly prolonged the survival time and significantly increased the survival rate of aGVHD mice, and the effect of CD90⁺hAECs treatment group was superior to that of the unscreened hAECs treatment group.

FIG. 5 Graphical representation of the survival rate of mice in each group over time. All mice in the model group died. Compared with unselected hAECs cells, CD90+ hAECs cells had better therapeutic effect and lower mortality rate.

FIG. 6 Flow cytometry shows the proportion of Th1, Th2 and Treg in mice in each group. FIG. 6-1 shows a flow representative and statistical plot of the ratio of human Th1 (CD4+IFNγ+,) Th2 (CD4+IL4+), and Treg (CD4+CD25+FOXP3+)) to CD4+ cells in FACS-assayed mice two weeks after transplantation (n=5.) The results showed better immunomodulatory, inflammation-reducing effects of CD90⁺ hAECs cells compared to unscreened hAECs cells. FIG. 6-2 shows that the CD90+hAECs transformed T-cell subset in vivo and affected CD4+ T-cell activation were studied by flow cytometry and the results showed that the aGVHD disease group had a higher proportion of Th1 and a lower proportion of Treg. In the treatment group co-transplanted with CD90+hAECs at the same time, the Th1 ratio decreased significantly. Importantly, we detected a nearly 4-fold increase in the Treg ratio. In the unscreened hAECs treatment group, we saw a more obvious suppression of the Th1 ratio compared with the CD90+hAECs treatment group. In addition, we also found that the proportion of Th2 subset did not change significantly regardless of the CD90+hAECs treatment group or the unscreened hAECs treatment group. The above results showed that CD90+hAECs improve aGVHD by changing the proportion of T cell subsets.

FIG. 7 Illustration of endothelial adhesion molecule expression in various groups of mice by tissue immunofluorescence. The results showed that compared with the negative control group (i.e., PBS injection group), the expression of adhesion molecules I-CAM1 and V-CAM1 on the vascular endothelium of the aGVHD group implanted with PBMCs was significantly increased, and as we expected, CD90+hAECs Co-transplantation with PBMC significantly weakened the expression of endothelial I-CAM1 and V-CAM1.

FIG. 8 Illustration of HE staining of target organ sections of mice in each group. The target organs from the mice with aGVHD disease: liver, lung, small intestine, kidney (12 days after PBMC injection) were taken and sectioned. HE staining showed that large-scale endothelial inflammation foci appeared in the hepatic portal vein of mice in the GVHD model group. The alveoli of the lungs showed inflammatory cell infiltration and peri-endothelial necrotic nodules. The kidneys showed localized edema. Blunting of small intestinal villi was found in the small intestine. While in the PBMC and CD90+hAECs simultaneous transplantation group, we found that liver endothelial inflammation foci almost disappeared. The infiltration of alveolar inflammatory cells was significantly reduced, and the necrotic area around the endothelium was significantly reduced. No obvious lesions were found in the kidneys and small intestine. In the unscreened hAECs group, we found that the therapeutic effect on liver and lung lesions was not as strong as in the CD90⁺ hAECs group, but a significant reduction in the extent of the lesions was still observed compared with the disease group. However, the treatment group with unscreened hAECs did not have a significant effect on renal pathologic changes, and the improvement in the small intestine was as obvious as in the CD90+hAECs group.

FIG. 9 Illustration of pathological scores of mouse organs in each group.

FIG. 10 Illustration of Masson staining of mouse lungs in each group. The results showed that the CD90⁺hAECs treatment group was significantly better than the unselected hAECs treatment group on Masson staining indicating fibrosis.

FIG. 11 Illustration of lentiviral infection of hAECs. Pictures of hAECs infected with GFP-tagged lentivirus under bright field (11a) and green fluorescence microscopy (11b) showed a high infection rate.

FIG. 12 Illustration of the localization of hAECs in mice. The results showed that CD90+hAECs were mainly located in the kidney, lung, and liver.

### DETAILED DESCRIPTION OF THE INVENTION

The present invention provides a method of treating a subject suffering from graft-versus-host disease (GvHD) or at risk of suffering from graft-versus-host disease, generally comprising administering to the subject human amniotic membrane epithelial cells (hAECs) that are effective to ameliorate at least one symptom or clinical sign of the graft-versus-host disease as compared to a suitable control subject. In order to further improve the clinical treatment effect, a cell subset of CD90⁺ hAECs with potent immunomodulatory capabilities is selected as the primary therapeutic cells.

In one aspect, the present invention discloses the use of human amniotic epithelial cells or cell preparations thereof in treating and/or improving graft-versus-host disease, wherein said human amniotic epithelial cells (hAECs) is CD90⁺ hAECs cell subset. An effective doses of CD90⁺ human amniotic epithelial cells or cell preparations thereof can be used alone or in combination with other drugs to treat and/or improve graft-versus-host disease. An effective dose is an amount sufficient to ameliorate or prevent the symptoms or conditions of a medical disease. The effective amount for a particular subject will vary depending on factors such as the disease being treated, the general health of the patient, the method, route and dosage of administration, and the severity of side effects. An effective amount may be the maximum dosage or dosage regimen that avoids significant side effects or toxic effects.

In another embodiment of the present invention, the CD90⁺hAECs transformed T cell subsets were studied in vivo by flow cytometry for clustering and affecting CD4+ T cell activation, and the results showed that the aGVHD disease group had a higher proportion of Th1, Th17, and a lower proportion of Treg. In the treatment group with simultaneous cotransplantation of CD90+hAECs showed a more pronounced decrease in the proportion of Th1, and importantly we detected an almost significant increase in the proportion of Treg compared to the unscreened hAECs treatment group. Importantly, we detected a nearly 4-fold significant increase in the proportion of Treg, while in the CD90+hAECs treatment group we saw a more pronounced suppression of the proportion of Th1 compared to the unscreened hAECs treatment group. In addition, we also found that the proportion of Th2 subset did not change significantly in either the CD90+hAECs or unscreened hAECs groups. These results suggest that CD90+hAECs improve aGVHD by shifting the proportion of T cell subsets and have more therapeutic value than unscreened hAECs.

In another embodiment of the present invention, the animal suffering from graft-versus-host disease refers to a mammal. In a more preferred embodiment, the animal is cow, horse, sheep, monkey, dog, rat, mouse, rabbit or human. In the most preferred embodiment, the animal suffering from graft-versus-host disease refers to a human being. In one embodiment of the present invention, the implantation of CD90⁺hAECs, in addition to improving the quality of survival of the diseased mice, more importantly prolongs and significantly improves the survival of the aGVHD mice, and likewise the CD90⁺hAECs treatment group has a better effect than the unscreened hAECs treatment group.

In another embodiment of the invention, said cell preparation comprises CD90⁺ Human amniotic epithelial cells and a pharmaceutically acceptable carrier. The pharmaceutically acceptable carrier described in the present invention refers to a substance that is suitable for human and/or animals and has an appropriate benefit/risk ratio without excessive adverse side effects (such as toxicity, irritation and allergic reaction), such as a pharmaceutically acceptable solvents, suspending agents or excipients, that are favorable for cell survival, and that are capable of delivering the formulated cells to the human or animal. The carrier is selected to suit the intended mode of administration. The carriers of the present invention include, but are not limited to, various physiological buffers such as saline, phosphate buffer, artificial cerebrospinal fluid or whole blood serum, umbilical cord serum, and the like.

CD90⁺ amniotic epithelial cells can be administered to the patient using any suitable method, such as intravenous injection or intraspinal injection. Typically these cells are contained in a pharmaceutically acceptable liquid culture medium. Administration of cells can be repeated or continuous (eg, by continuous infusion into the cerebrospinal fluid). In generally, multiple dosing regimens are usually administered separately at intervals of at least 7-10 days. An alternative approach is to plant cells in a bioabsorbable material such as gelatin sponge, and use surgery to implant the bioabsorbable material seeded with cells into the desired site. The above two methods can be combined to achieve better results.

The appropriate dosage of CD90+ amniotic epithelial cells will vary based on the patient's age, gender, weight, health status, and other factors. Typically, the dosage range per administration is about 10³-10⁹ cells, typically about 10⁶-10⁸ cells.

In another embodiment of the invention, the present invention discloses use of human amniotic epithelial cells or cell preparations thereof in the preparation of drugs for treating and/or improving graft-versus-host disease, wherein said human amniotic epithelial cells (hAECs) is CD90⁺ hAECs cell subset.

In another embodiment of the present invention, the invention discloses the use of CD90⁺ human amniotic epithelial cells or cell preparations thereof in combination with other drugs to treat and/or improve graft versus host disease. CD90+ amniotic epithelial cells are administered to the patient together with one or more drugs selected from the group consisting of methylprednisolone, cyclosporine, tacrolimus, mecophenolate mofetil, methotrexate, glucocorticosteroids, azathioprine, thalidomide, an anti-T-cell monoclonal antibody (anti-CD3 monoclonal antibody), an antibody against the interleukin-2 receptor, etc., or combinations thereof.

In another embodiment of the invention, the invention discloses a method for isolating amniotic epithelial cells from amniotic tissue, comprising the following steps:
(1) Obtain amniotic membrane from placental tissue through mechanical separation;
(2) The amniotic membrane is washed and then digested with digestive enzymes, and the digested liquid is centrifuged to obtain human amniotic membrane epithelial cells;
(3) Sort out CD90⁺ human amniotic epithelial cells (hAECs).

In another embodiment of the present invention, the amniotic membrane epithelial cells described herein are derived from human. The amniotic membrane can be isolated from separated human placentas by rinsing with physiologic buffer to remove blood cells and mechanically removing residual chorionic villi and blood vessels. “Isolation” refers to removing cells from a tissue sample and separating them from another tissue. Single cells are isolated from intact human amniotic epithelial tissue using any conventional technique or method, including mechanical force (mincing force or shearing force), enzymatic digestion with one or a combination of proteases such as collagenase, trypsin, lipase, liberase, and pepsin or a combination of mechanical and enzymatic methods.

In another embodiment of the present invention, the screening process of CD90+ human amniotic epithelial cells is as follows: hAECs are stained with CD90 primary antibody, then incubated with fluorescence-conjugated secondary antibody, and analyzed by flow cytometry to sort out CD90⁺ human amniotic epithelial cells. A more preferred screening process is as follows: hAECs are stained with CD90 primary antibody(1:20, Millipore), and then incubated with fluorescence-conjugated secondary antibodies. FACS Calibur instrument (Becton Dickinson, Franklin Lakes, NJ, USA) Flow cytometry analysis is performed to sort out CD90+ human amniotic epithelial cells.

In a preferred embodiment of the present invention, the human amniotic membrane shall be obtained with the authorization and consent of the puerpera. The placental tissue of a healthy puerpera after caesarean section is taken and the entire amniotic membrane is obtained through mechanical separation.

In another preferred embodiment of the present invention, the CD90⁺ human amniotic epithelial cells obtained in step 3 can be continued to be cultured, and the preferred culture conditions are as follows: inoculate the cells in a petri dish at a density of 1×10⁶-1×10⁸ cells/plate, place the cells in a carbon dioxide incubator for cultivation, and then change the culture medium after the CD90⁺ human amniotic epithelial cells adhere to the wall, and the cells are digested and frozen after the cells cover the plate.

The active cell population can be concentrated by other methods known to those skilled in the art. These post-processing washing/concentrating steps can be performed individually or simultaneously. In addition to the above methods, the active cell population can be further purified or enriched to reduce stray cells and dead cells after the cells are washed or cultured. Isolation of cells in suspension can be accomplished by the following techniques: buoyant density sedimentation centrifugation, differential adhesion to and elution from the solid phase, immunomagnetic beads, fluorescent laser cell sorting (FACS), or other techniques. Examples of these different techniques and devices for performing them can be found in the prior art and commercially available products.

There is no limitation on the type of basal medium used in the present invention as long as the medium can be used for cell culture. Preferred media include DMEM media and NPBM media. There are no restrictions on the types of other components that may be contained in the above-mentioned basal culture medium. Preferred components include F-12, FCS, neural survival factor, and the like.

In another preferred embodiment of the present invention, bFGF (basic fibroblast growth factor) or EGF (epidermal growth factor) is added to the basal medium above-mentioned. In this case, one or both may be added. Exemplary concentrations of the bFGF or EGF above-mentioned are from 1 ng/ml to 100 ng/ml, preferably at a concentration of 10 ng/ml. There are no restrictions on when and how to add. Preferably, the reagents are added daily while culturing the above-mentioned amnion epithelial cells in the basal medium.

The present invention uses an effective dose of CD90⁺ amniotic epithelial cells or a cell preparation containing CD90⁺ amniotic epithelial cells alone or in combination with other drugs to treat and/or improve graft versus host disease. The present invention investigates the potential ability of CD90⁺ human amniotic epithelial cells in the treatment of graft-versus-host disease and explores its therapeutic mechanism. The results show that both the CD90⁺ hAECs and PBMC co-transplanted treatment group and the unscreened hAECs treatment group have a better inhibitory effect on aGVHD, which significantly improves the clinical and pathological phenotypes of the mice compared to the disease group, as well as significantly improves the survival rate of the mice. Using the animal model of the present invention, it is demonstrated that the method can effectively reduce the infiltration of inflammatory cells into target organs caused by aGVHD, as well as significantly reduce target organ lesions. hAECs are also found to have a pro-apoptotic effect on a number of leukemia cell lines, and can be used for the treatment of graft-versus-host disease, which will have broad prospects in clinical application for the treatment of graft-versus-host disease.

The present invention uses CD90⁺ human amniotic epithelial cells for the treatment of graft-versus-host disease, which fully utilizes the advantages of human amniotic epithelial cells, in which human amniotic epithelial cells have the following main advantages:
(1) It can maintain pluripotency for a long time and has the unique potential of embryonic stem cells to differentiate into three germ layer tissues;
(2) MHC type II molecules are hardly expressed on the cell surface and therefore do not cause inflammatory, allergic and immune reactions, and the requirements for graft matching are reduced accordingly;
(3) It has the ability to modulate the immune response in vivo and in vitro, and secretes a variety of immunomodulatory factors, antiangiogenic proteins, or anti-inflammatory factor-related proteins when cultured in vitro;
(4) It has low immunogenicity and can be regarded as immune amnesty cells without the function of antigen presentation, which can reduce the source of immune cells after transplantation and avoid the occurrence of immune rejection;
(5) It does not express telomerase reverse transcriptase and has no tumorigenicity (including benign tumors, sarcomas and carcinomas);
(6) It has a strong proliferative capacity and is able to maintain a vigorous proliferation in the previous generation (within about 10 generations);
(7) It has a wide range of sources, easy to obtain, no application restrictions, and no ethical issues.

### EXAMPLE

### Example 1 Isolation and Culture of Primary Amniotic Epithelial Cells

### 1. Sources of Human Amniotic Membrane

In order to avoid microbial contamination of the birth canal, we chose to use the placenta of a cesarean sectioned fetus. Due to the stimulation of labor signals after term, the amniotic membrane would undergo apoptosis, so it was appropriate to use the Preterm fetal placenta (before 38 weeks). After authorized consent from the puerpera, placental tissue from a healthy mother (whose serological reactions are negative for HIV, syphilis, hepatitis A, hepatitis B, and hepatitis C) after caesarean section was taken, the placenta was cut with a cross knife, and the whole amniotic membrane was obtained by mechanical separation.

### 2. Isolation and Sorting of CD90⁺hAECs (Aseptic Operation is Required Throughout the Process)

The placenta of an infant born by cesarean section before 38 weeks was obtained, and the amniotic membrane was peeled from the inner surface of the placenta and immersed in a centrifuge tube containing F12/DMEM (containing 1× penicillin streptomycin and amphotericin) basal medium. Samples are transported at 4° C. cold-chain to the laboratory cellular compartment.

The amniotic membrane was removed and each amniotic membrane was washed in 40 ml of CMF-HBSS (containing 1× penicillin-streptomycin and amphotericin) to remove mucus, and the mesenchymal layer and mucus close to the chorionic layer were scraped off with forceps, and the procedure was repeated three times, with a new container and new HBSS solution for each wash.

The washed amniotic membrane was transferred into a new container, added 10 ml 0.05% trypsin/EDTA, inverted for 30 s, and the solution was discarded.

The amniotic membrane was transfer into a new container, added 20 ml 0.05% trypsin/EDTA, incubated at 37° C. for 10 min and the solution was discarded.

The amniotic membrane was transferred to a new container and incubated with 25 ml trypsin/EDTA at 37° C. for 40 min, and the digestive solution was preserved.

After the initial digestion, the amniotic membrane was transferred to a new container and incubated with 25 ml of trypsin/EDTA in a 37° C. water bath for 40 min, and the digestive solution was preserved.

An equal volume of digestion termination solution (F12/DMEM with 5% FBS, 1× L-glutamine, 1× β-mercaptoethanol, 1× pyruvic acid) was added and centrifuged at 400 g for 10 min. The supernatant was discarded, and the precipitate was resuspended with amniotic complete medium: F12/DMEM containing 5% KSR (KnockOut Serum Replacement), 1× L-glutamine, 1× β-mercaptoethanol, 1× pyruvic acid, 1Xps (Penicillin-Streptomycin), 10 ng/ml hEGF.

Added an equal volume of digestion termination solution and centrifuged at 400 g for 10 min. The supernatant was discarded and resuspend the precipitate in complete medium.

Passed through a 100 um sieve and counted.

Washed with PBS.

Flow cytometry analysis was performed with a FACS Calibur instrument (Becton Dickinson, Franklin Lakes, NJ, USA). The hAEC were stained with fluorescent coupled antibodies targeting CD34, CD45, CD31, CD29, CD166, CD90, HLA-DR, and HLA-DQ using standard protocols (1:20, all from BioLegend). For SSEA4 and TRA-1-60, hAEC were stained with SSEA4 (1:20, Millipore) and TRA-1-60 (1:20, Millipore) primary antibodies and then incubated with fluorescently coupled secondary antibodies. Measurements of cell cycle distribution were obtained using propidium iodide (PI) staining. Isotype controls were used in each experiment.

Setting up the FACS Calibur instrument program (Becton Dickinson, Franklin Lakes, NJ. (USA) for flow cytometric sorting of CD90-positive human amniotic epithelial stem cells. The flow rate and sample concentration were adjusted so that the target cells passed through the sample chamber at a concentration of 100-300 cells/second, and CD90-positive human amniotic epithelial stem cells were screened and collected.

Cells were inoculated into petri dishes at 10\hat{ }5 cells/cm² or frozen in liquid nitrogen in freezing solution (90% FBS, 10% DMSO) for later use.

3. Inoculation Culture and Freezing of hAECs

Cell counting culture:

$$Concentration = \frac{total number of cells in 4 cells}{4} \times 10 , 000 \times dilution ( cells / ml ) ,$$

a plates was inoculated with 1×10⁷ cells. The culture medium was changed after the hAECs had adhered to the wall, and then the culture medium was changed once every three days.

After the cells had grown all over the plate, the cells were digested and frozen: 15 cm dish with 5 ml pancreatic enzyme, 10 mins later, the cells were observed under the microscope, and the digestion was terminated by adding an equal amount of digestion termination solution when the cells became rounded and suspended when the dish was shaken on a flat surface. Blow down the cells on the petri dish with a micropipette in the same direction, transferred them into a 15 ml centrifuge tube, centrifuged at 300 g for 3 mins, collected the cells and then counted them. After added freezing solution into the freezing tube, labeled the date, batch and number of cells, the cells were put into the freezing tube, and then immediately put the freezing tube into a freezer box and put the freezer box into a −80° C. refrigerator, and then taken out the freezer box after 12 h and transferred the cells into liquid nitrogen tanks for preservation

### Example 2 Human Peripheral Blood Mononuclear Cell Isolation, Culture and Freezing

Aseptically draw the donor's peripheral blood into a collection strip containing anticoagulant.

Added 5 ml of Ficoll's solution to a 50 ml centrifuge tube, draw blood and gently drop it into the Ficoll's solution using a sterile pasteurized pipette.

Without centrifugal acceleration, centrifuged the blood at 400 g for 30 min at room temperature until the blood appears to be clearly stratified.

Carefully aspirated the white membrane layer from the stratification with a new pasteurized pipette and added it to 10 ml DPBS (containing 1×PS) and mix well.

Centrifuged at 300 g for 5 min at room temperature and the supernatant was discard.

The precipitate was resuspended in 10 ml of DPBS (containing 1×PS), centrifuged at 300 g for 5 min at room temperature and the supernatant was discard.

The precipitate was resuspended with complete medium (1640, 10% FBS, PS) and set aside, or resuspended with freezing solution and freeze in liquid nitrogen.

### Example 3 Mouse Model Establishment and aGVHD Mouse Scoring Criteria

### 1. Establishment of Acute GVHD Model in NCG Mice

NCG mice aged 6-8 weeks were purchased from the company and transferred to the animal house for 1 week to acclimatize. Gentamicin (32×10\hat{ }4 U/L) and erythromycin (250 mg/L) were added to the drinking water.

The experimental mice were divided into GVHD model group, CD90⁺hAECs treatment group, unscreened hAECs treatment group and control group, n=5. Mice in the GVHD model group, CD90⁺hAECs treatment group and unselected hAECs treatment group were injected with 10\hat{ }7 human peripheral blood mononuclear cells (PBMC)/mouse via the tail vein. One week later, mice in the CD90⁺hAECs treatment group were injected into CD90⁺hAECs 2×10\hat{ }6/mouse via the tail vein, unselected hAECs treatment group were injected into unselected hAECs 2×10\hat{ }6/mouse via the tail vein. The control group was injected with the corresponding volume of PBS.

2. aGVHD Mouse Scoring Criteria

aGVHD mouse clinical phenotype scale (based on the KR. Cook scoring system, 10 points, observed every other day).

Weight loss: mice with weight loss less than 10% were considered normal and scored 0; weight loss between 10% and 25% was scored 1 point; weight loss greater than 25% was scored 2 points.

Posture changes: 0 points for mice with no abnormal posture; 1 point for mice with an arched back only at rest. 2 points for severe arching of the back that interferes with normal activities.

Changes in hair texture: mice with no abnormal hair texture were scored 0 points as normal; 1 point for slight wrinkles; 2 points for severe wrinkling.

Activity: mice scored 0 for normal activity, 1 for slightly reduced activity, and 2 for inactive if no stimulation.

Skin integrity: 0 points for mice with no skin abnormalities recognized as normal; 1 point for skin flaking on the tail or paw; 2 points for visible areas of flaking skin.

Scoring Criteria for Organ Pathology in aGVHD Mice (4 Points Total)

Lung: Normal without lesions was considered normal and was scored as 0 points; a small amount of leukocyte accumulation around blood vessels was scored as 0.5 points; the degree of perivascular leukocyte aggregation was 1-2 cells thick (involving 15% of the perivascular region), scored as 1 point; the degree of perivascular leukocyte aggregation was 1-2 cells thick (involving 15% of the perivascular region) and infiltrated into parenchymal tissue, scored as 1.5 points; the degree of perivascular leukocyte accumulation was 2-3 cells thick ((involving 15% of the perivascular region) and infiltrated into parenchymal tissue, scored as 2 points; the degree of perivascular leukocyte aggregation was 2-3 cells thick (involving 25%-50% perivascular region) and infiltrated into parenchymal tissue, scored as 2.5 points; the degree of perivascular leukocyte aggregation was 4-5 cells thick (involving 25%-50% perivascular region) and infiltrated into the parenchymal tissue, scored as 3 points; The degree of perivascular leukocyte aggregation is 6-7 cells thick (involving 50% of the perivascular region) and infiltrated into the parenchymal tissue, seriously damaged the normal structure of the tissue, scored as 3.5; The degree of perivascular leukocyte aggregation was 6-7 cells thick (involving more than 50% perivascular region) and infiltrated into parenchymal tissue, seriously damaged the normal structure of the tissue, scored as 4;

Intestine: Normal and without lesions was considered normal and scored as 0 points; necrosis of pit cells occasionally occurs, and very few inflammatory cells infiltrated into the submucosa and small intestinal villi, scored as 0.5 points; necrosis of pit cells reached 15%, and inflammatory cells infiltrated into 20% of the submucosa and small intestinal villi, scored as 1 point; the necrosis of the pit cells reached 15%, and the inflammatory cells infiltrated into one-third of the submucosa and the small intestinal villi, scored as 1.5 points; the necrosis of the pit cells reached 25%, and inflammatory cells infiltrated into ⅓ of the submucosa and the small intestinal villi, scored as 2 points; necrosis of pit cells reached 25%-50%, and inflammatory cells infiltrated into ⅓ of the submucosa and small intestinal villi, scored as 2.5 points; necrosis of pit cells was greater than 50%, inflammatory cells infiltrated into ⅓ of the submucosa and small intestinal villi, scored as 3 points; necrosis of the pit cells was greater than 50%, and inflammatory cells infiltrated into 50% of the submucosa and small intestinal villi, scored as 3.5 points; necrosis of the pit cells was greater than 50%, and inflammatory cells infiltrated into more than 50% of the submucosa and small intestinal villi, scored as 4 points.

Spleen: Normal and without lesions was considered normal and scored as 0 points; 10 necrotic or apoptotic cells per square millimeter of tissue was scored as 1 point; 10 necrotic or apoptotic cells per square millimeter of tissue with occasional hemolysis was scored as 1.5 points; There were 10-20 necrotic or apoptotic cells per square millimeter of tissue, with occasional hemolysis accompanied by the tissue structure damage, scored as 2 points; there were 10-20 necrotic or apoptotic cells per square millimeter of tissue, with hemolysis accompanied by less than 25% tissue structure damage, scored as 2.5 points; there were 20-40 necrotic or apoptotic cells per square millimeter of tissue, with hemolysis accompanied by 25%-50% tissue structure damage and less than 25% tissue fibrosis, scored as 3 points; there were 20-40 necrotic or apoptotic cells per square millimeter of tissue, hemolysis accompanied by 50% tissue structural damage and 25%-50% tissue fibrosis, scored as 3.5 points; there was extensive necrotic or apoptotic cell death (more than 50% area of tissue), with hemolysis accompanied by more than 50% tissue structural damage and more than 50% tissue fibrosis, scored as 4 points.

Liver: Normal without lesions was considered normal and scored as 0 points; parenchymal tissue in the lesion area accumulated 1-2 mononuclear cells per 0.5 cm of tissue, scored as 1 point; there was one endothelial blood vessel per 0.5 cm of tissue, and had at least 2 inflammatory cells infiltrating the subendothelium of each blood vessel, scored as 2 point; there was 3 endothelial blood vessel per 0.5 cm of tissue, and had at least 3 inflammatory cells deep infiltrating the subendothelium of each blood vessel, scored as 3 point; Endothelitis was present in almost all blood vessels, and deep infiltration of at least 3 inflammatory cells under the endothelium of each vessel, scored as 4 points.

### Example 4 Experimental Grouping of aGVHD Mouse Model

NCG mice without mature T cells, B cells, and NK cells in the body were used as model mice, and human peripheral blood mononuclear cells (PBMC) were implanted to establish a humanized aGVHD mouse model. We divided the experiment into four groups: 1. PBMC transplant alone group as aGVHD disease model group; 2. CD90⁺hAECs and PBMC transplanted simultaneously as aGVHD disease CD90⁺hAECs treatment group; 3. unscreened hAECs transplanted simultaneously with PBMC as aGVHD disease unscreened hAECs treatment group; 4. Injection of the corresponding volume of culture fluid as a blank control group.

| Groups | Model Building |
|---|---|
| aGVHD | PBMC |
| aGVHD Treating | PBMC & CD90 + hAECs |
| aGVHD Treating | PBMC & hAECs |
| Control | PBS |

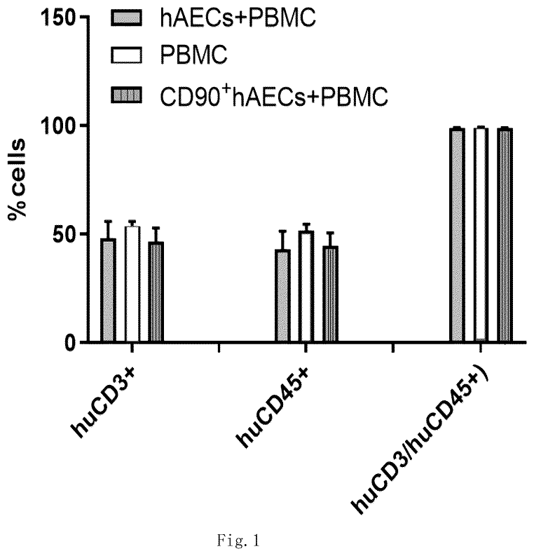

The results showed that 2 weeks after PBMC implantation, FACS results indicated that CD3 and CD45 (CD45 molecules were expressed on all leukocytes and chimerism was up to more than 50% in aGVHD model mice (FIG. 1.). While implanted at the same time either CD90⁺ hAECs or unselected hAECs did not significantly affect PBMC implantation (FIG. 1.) In addition, we found that almost all implanted CD45⁺ cells co-expressed CD3 (FIG. 1.)

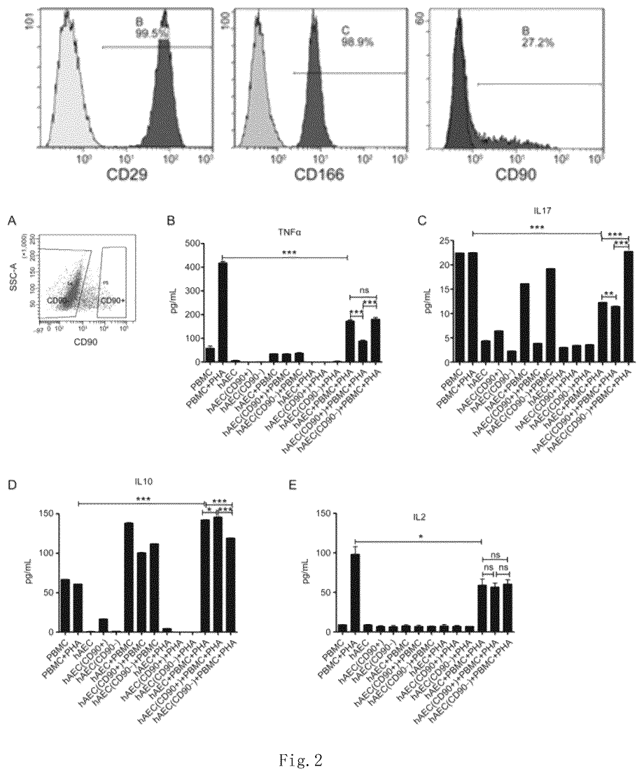

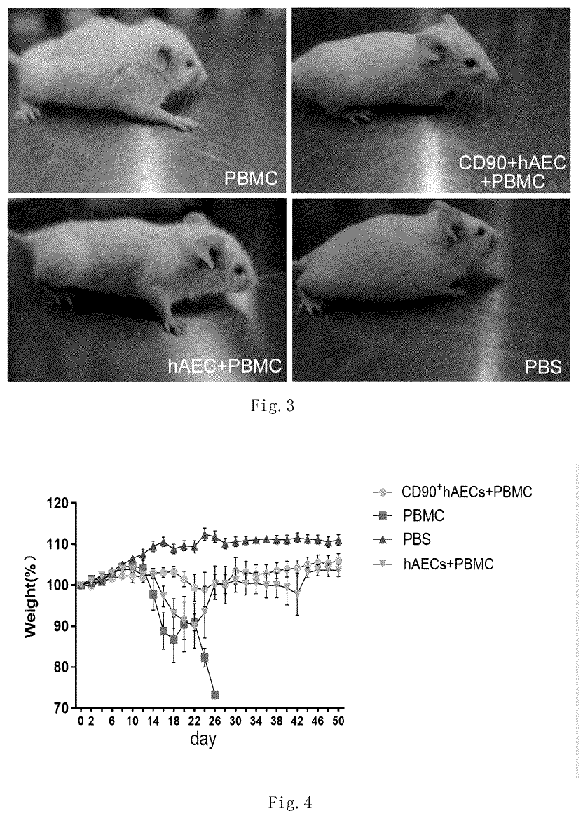

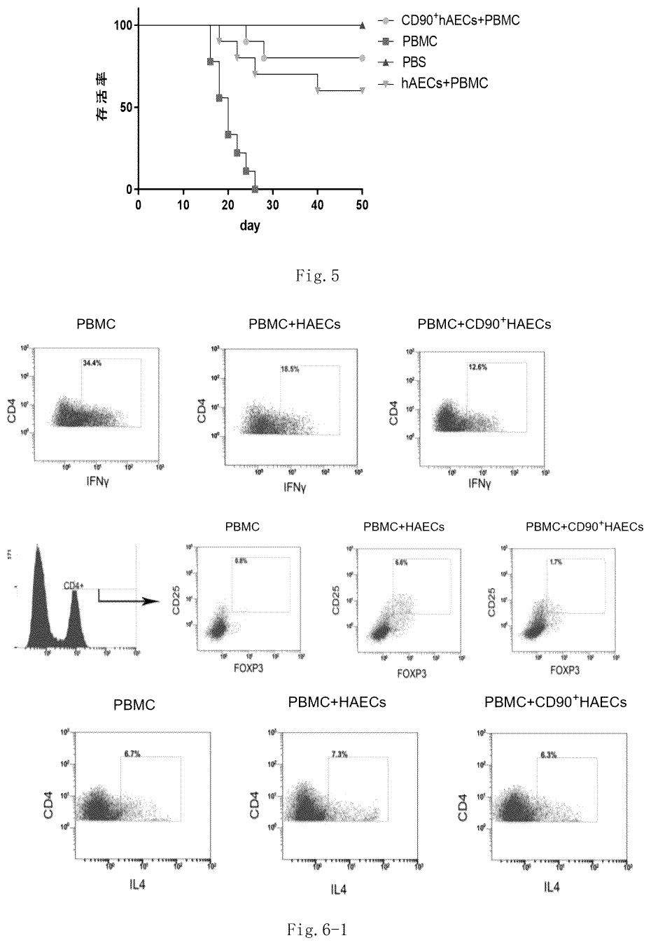

Two weeks after PBMC implantation, we observed that mice implanted with PBMC alone gradually developed a series of clinical signs typical of aGVHD: weight loss, decreased mobility, depression, severe bowed backs, hair loss, diarrhea, etc., whereas mice co-transplanted with CD90⁺hAECs (i.e., the CD90⁺hAECs treatment group) showed a very significant improvement of all the disease signs. In the unscreened hAECs group, although the treatment effect was not as good as that of the CD90⁺hAECs group, there was still a significant improvement compared with the disease group. However, in terms of bowed back, diarrhea (which was consistent with the tissue staining later), and mobility, the unscreened hAECs group did not differ significantly from the CD90⁺hAECs group (FIGS. 2 and 3). We performed clinical scoring of this phenotype, and the results showed significant differences between the CD90⁺hAECs treatment group & unscreened hAECs groups and the disease group.

In addition to improving the quality of life of diseased mice, more importantly the implantation of CD90⁺hAECs extended the survival time of aGVHD mice and significantly improved their survival rate. Similarly, the effect of CD90⁺hAECs treatment group was better than that of unscreened hAECs treatment group (FIG. 4).

### Example 5 Flow Cytometry

### 1. Cell Surface Marker Staining

Erythrocyte lysis was required if the sample is tissue or blood cells. Added 3 ml of red blood cell lysis buffer (ACK) to the cells in the tube to resuspend the cells, and incubated on ice for 5 to 20 minutes.

Added 10 ml of cell staining solution (2% FBS in PBS) to the tube to terminate ACK lysis. Centrifuged at 350 g for 5 min at room temperature and discarded the supernatant. Repeated lysis with ACK if necessary.

Repeated step 2 to wash the cells once.

After viable cell counting, added cell staining solution to adjust the cell number concentration at 5-10×10\hat{ }6 cells/ml, and dispense 100 ul of cell solution per tube into flow cytometry tubes.

Added an appropriate amount of primary antibody coupled with a fluorescent marker to the prepared cell suspension. Incubated on ice in the dark for 20-30 minutes.

The cells were washed twice with 2 ml of Cell Staining Solution, centrifuged at 350 g for 5 min at room temperature, and the supernatant was discarded.

Added 0.5 ml Cell Staining Buffer to resuspend the cells and analyzed by flow cytometry.

### 2. Intracellular Factor Staining

Stimulus-activated cells were collected, which could be either tissues stimulated in vivo or cultured cells stimulated in vitro. During the last 4-6 hours of the stimulation process, the protein transport inhibitors brefeldin A or monensin are added to inhibit cytokine secretion into the extracellular compartment. Stained tubes as needed.

Surface staining of live cells were performed prior to fix rupture of membrane and intracellular staining.

Prior to intracellular staining, cells were fixed with Fixation Buffer, 0.5 ml of Fixation Buffer per tube, and incubated at room temperature away from light for 20 min.

Centrifuged at 350 g for 5 minutes and discarded the supernatant.

If there was a need to abort the experiment, paused and saved at this step to prepare for continuing the experiment later. Cells were washed with Cell Staining Buffer, centrifuged at 350 g for 5 minutes, and the supernatant was discarded. Cell Staining Buffer was used to resuspend the cells for short-term storage at 4° C. The cells may be stored at −80° C. for longer periods of time with 90% FCS/10% DMSO.

Fixed cells were resuspended with Permeabilization Wash Buffer and centrifuged at 350 g for 5-10 minutes, discarded supernatant.

Repeated step 6.

Fixed cells were resuspended with 100 ul Permeabilization Wash Buffer, added specific fluorescent labeling antibody (the amount of antibody should be added according to the instruction), and protected the cells away from light for 20 min at room temperature.

Cells were washed twice with 2 ml Permeabilization Wash Buffer. Centrifuged at 350 g for 5 minutes, discarded supernatant.

The fixed stained cells were resuspended with 0.5 ml Cell Staining Buffer and analyzed by flow cytometry.

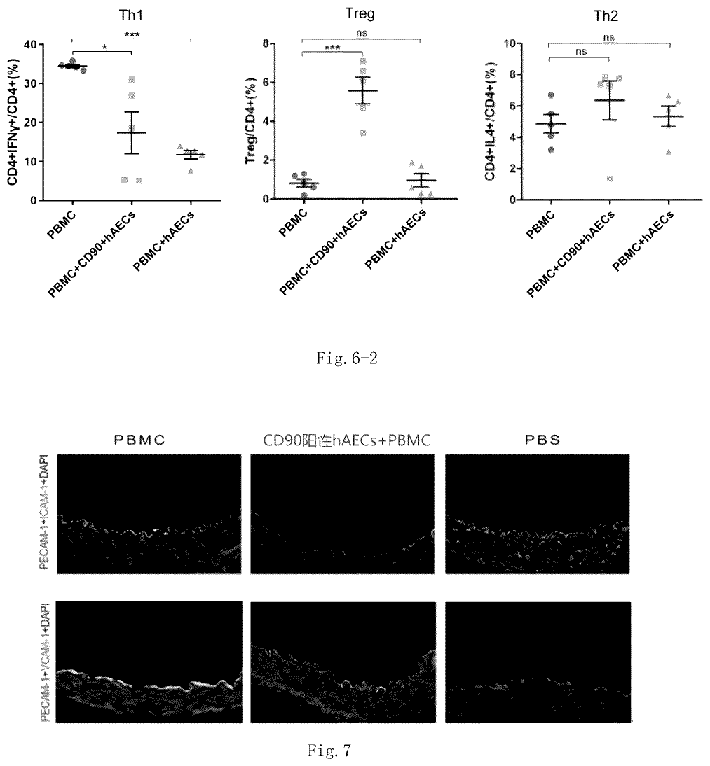

Flow cytometry was used to study how CD90⁺ hAECs transformed T cell subsets in vivo and affected CD4⁺ T cell activation. The results showed that the aGVHD disease group had a higher proportion of Th1, Th17 and a lower proportion of Treg (FIG. 6). In the treatment group transplanted simultaneously with CD90⁺ hAECs, the Th1 proportion decreased significantly. What was important was that we detected a nearly 4-fold increase in the Treg proportion (FIG. 6). In the CD90⁺hAECs treatment group, we found that the proportion of Th1 cells was more significantly compared to the unselected hAECs treatment group (FIG. 6). In addition, we also found that the proportion of Th2 subset did not change significantly in either the CD90⁺hAECs-treated or unscreened hAECs-treated groups (FIG. 6). The above results showed that CD90⁺ hAECs improve aGVHD by changing the proportion of T cell subsets.

### Example 6 Immunofluorescence

### 1. Cellular Immunofluorescence

Aspirated the culture medium from each well of the cells (24-well plate), added 1 ml of PBS, shook and washed, and then discarded the liquid.

Added 1 ml of 4% paraformaldehyde (diluted in PBS) and incubated at room temperature for 30 min.

Discarded the solution, shook and washed with PBS twice and discarded the solution.

Added 500 ul of 0.2% Triton X-100 (diluted in PBS) and incubated at room temperature for 15 min (skip this step if the antigen is a membrane protein).

Discarded the solution, shook and washed with PBS twice and discarded the solution.

Added 10% FBS (or 3% goat serum or 2% horse serum) to seal for 1˜2 h.

Discarded the liquid, added PBS, placed on a horizontal shaker at 60 rpm and washed for 5 min at room temperature. Repeated 3 times.

primary antibody was diluted with 10% FBS or 3% horse serum, and added 250 ul per well. Incubated at room temperature for 1 h (or overnight at 4°).

Discarded the liquid, added 500 ul wash buffer, placed on a horizontal shaker at 60 rpm and washed for 5 min at room temperature. Washed for 3 times.

The fluorescent coupling secondary antibody was diluted with 10% FBS or 3% horse serum and added 250 ul to each well. incubated at room temperature and away from light for 1 h (All subsequent steps starting from this step should be protected from light).

Discarded the liquid, added 500 ul wash buffer, placed it in a horizontal shaker at 60 rpm and washed for 5 min at room temperature. Washed for 3 times.

DAPI was diluted with PBS, added 250 ul per well and incubated at room temperature for 5 min.

Discarded the liquid, added 500 ul wash buffer, placed in a horizontal shaker at 60 rpm and washed for 5 min at room temperature, washed for two times.

Discarded the liquid, added 250 ul PBS to each well and inspected under microscope. If the cells were crawling, added the sealing solution to the coverslip, removed the crawler so that the cell side was facing the coverslip. Try not to generate bubbles in this step, and if bubbles were generated, used tweezers to squeeze out the air bubbles carefully, and then examined it under microscope.

### 2. Tissue Immunofluorescence

After the tissue was removed from the body, it is quickly washed in PBS to remove blood and carefully cut into appropriate sizes (be careful not to pinch the tissue). The tissue was placed in an embedding cassette containing an OCT. Placed the cassette in isopentane pre-cooled with dry ice until the OCT and tissue were completely frozen.

The embedded tissues were fixed in a frozen sectioning machine, cut into 0.5 um sections, and the sections were adhered to adhesive slides.

The embedded tissues were fixed in a frozen sectioning machine, cut into 0.5 um slices, and the slices were adhered to adhesive slides.

Slices should be left at room temperature for 30 min before staining, so that the slices and slides were fully adhered.

The samples were placed in acetone at −20° C. and fixed for 10 min (if the tissues were fixed before embedding, then proceeded directly to the subsequent operations).

Took out the slices and washed them three times with PBS for 5 minutes each time.

The tissue was first circled with an oil-based pen, and then the primary antibody was dropped on the tissue (the primary antibody was diluted in PBS with 5% HBS+1% BSA), and placed in a wet box at 4° C. overnight;

Took out the slices and washed them three times with PBS for 5 minutes each time.

Removed the moisture from the surface of the slices, added dropwise fluorescent-conjugated secondary antibodies (diluted with 5% HBS+1% BSA in PBS), and incubated in a wet box at room temperature in the dark for 1 hour. (Subsequent operations starting from this step should be protected from light)

Took out the slices and washed them three times with PBS for 5 minutes each time.

DAPI was diluted with PBS and added dropwise to the slices, incubated at room temperature for 3 min, and then washed twice with PBS for 5 min each time;

The slices were sealed with sealing solution and examined under fluorescence microscope.

We collected thoracic aortas from mice in each experimental and control group and performed frozen sections and immunofluorescence staining for adhesion molecules. The results showed that compared with the negative control group (i.e., PBS-injected group), the aGVHD group implanted with PBMC had significantly higher expression of adhesion molecules ICAM1 and V-CAM1 on the vascular endothelium, and as we expected, co-transplantation of CD90⁺hAECs and PBMCs significantly weakened the expression of endothelial I-CAM1 and V-CAM1 (FIG. 7). Therefore, we concluded that CD90⁺hAECs can inhibit the expression of endothelial adhesion molecules and thereby alleviate vascular GVHD.

### Example 7 Tissue Paraffin Embedding and Sectioning

After the tissues were removed from the body, they were quickly washed in 10% formalin to remove blood stains. A total of 3 washes were performed.

The tissues were fixed in 10% formalin or 4% PFA at room temperature.

The tissues were fished out, washed by PBS and dehydrated with 70% ethanol at room temperature.

After gradient dehydration with ethanol, the tissues were permeabilized in xylene.

The permeabilized tissues were paraffin-embedded and placed at 4° C. to solidify.

The paraffin blocks were placed in a microtome and cut into 5-10 μm slices according to different experimental needs.

After the steps of spreading the slices, fish slices and drying, the finished paraffin slices were obtained.

### Example 8 HE Staining

The paraffin slices were placed at 65° C. for 1 h for deparaffinization.

Slices were dewaxed in xylene solution three times and soaked for 10 min, 10 min, and 5 min sequentially.

The slices were taken out and hydrated in 100% ethanol for 5 min, 100% ethanol for 5 min and 95% ethanol for 2 min. The slices were then rinsed under running water for 5 min.

The slices were stained with hematoxylin for 5 min and then rinsed under running water for 5 min to remove excess dye.

The slices were soaked in ethanol hydrochloric acid solution for 2 s for differentiation, rinsed under running water for 5 min.

The slices were soaked in diluted ammonia for 6 s, rinsed under running water for 5 min, and then put into 95% ethanol for 4 min.

The slices were stained with eosin for 40 s.

The slices were put into 95% ethanol for 2 min, 95% ethanol for 2 min, 100% ethanol for 4 min, 100% ethanol for 4 min.

The slices were put into xylene solution for 5 min, 5 min, 5 min, respectively.

The slices were sealed with neutral resin and examined under microscope.

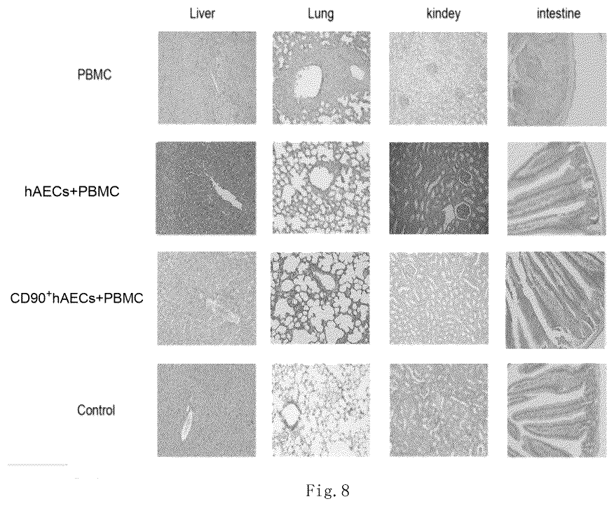

The results showed that large endothelial inflammatory foci appeared at the hepatic portal vein in the aGVHD model group of mice. In the lungs, alveoli were seen to be infiltrated by a large number of inflammatory cells, and necrotic nodules appeared around the endothelium. The kidneys showed localized edema. Blunting of small intestinal villi was found in the small intestine. In contrast, in the treatment group where PBMC were transplanted simultaneously with CD90⁺hAECs, we found that the endothelial inflammatory lesions in the liver almost disappeared. Alveolar inflammatory cell infiltration was significantly reduced, and the necrotic surface around the endothelium was significantly reduced. No significant lesions were found in the kidneys and small intestine. In the treatment group where PBMC were transplanted simultaneously with unscreened hAECs, We found that although the treatment of liver and lung lesions was not as good as that of the CD90⁺hAECs group, but still significantly reduced the degree of lesions compared with the disease group, and had no significant effect on renal pathological changes; and the improvement of the small intestine was as significant as that of the CD90+hAECs group, which was consistent with the small weight change and no diarrhea symptoms observed in the treatment group in the clinic (FIG. 8, FIG. 9).

### Example 9 Masson Staining

The paraffin slices were placed at 65° C. for 1 h for deparaffinization.

The slices were dewaxed in xylene solution three times and soaked for 10 min, 10 min, and 5 min sequentially.

The slices were taken out and hydrated in 100% ethanol for 5 min, 100% ethanol for 5 min and 95% ethanol for 2 min. The slices were then rinsed under running water for 5 min.

Nuclei were stained with Reagent A for 5 min and rinsed under running water for 5 min.

The slices were placed in an 65° C. oven until the water on the surface of the slide evaporated and the surface of slices turned white.

The slices were placed horizontally in a wet box, the specimen was covered with a drop of Reagent B and stained at room temperature for 10-20 min.

Added reagent C dropwise to the slices, incubated for 3 minutes, discarded the solution, and repeated twice.

Removed the moisture from the surface of the slices, added reagent D solution dropwise, and incubated for 3-5 minutes.

Aspirated off Reagent D, added reagent E and incubated for 5-15 seconds.

Added Reagent C aqueous solution dropwise and discarded the solution until the staining became clear.

The slices were dehydrated by ethanol gradient, permeabilized by xylene, sealed with neutral resin and examined under microscope.

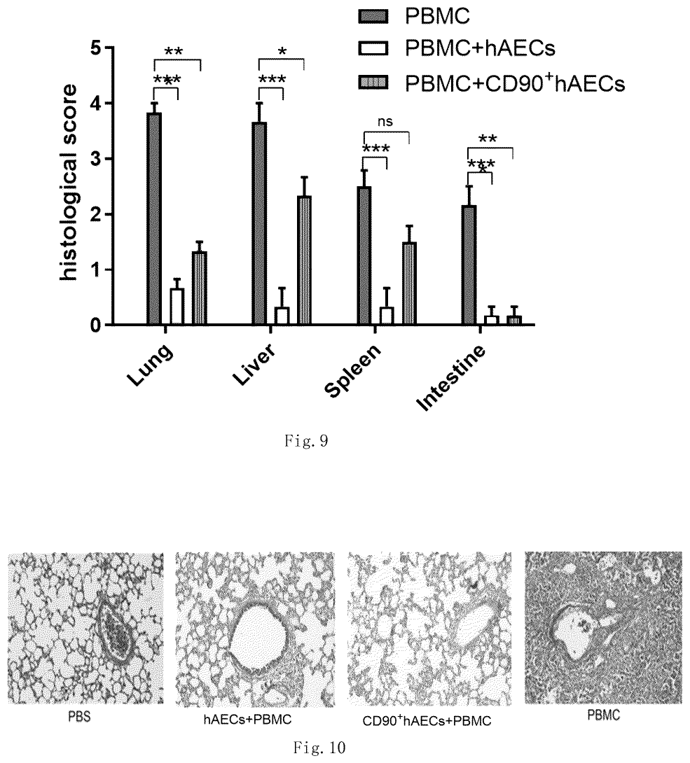

The results showed a large number of positively stained areas (blue) in the aGVHD model group, which suggested that fibrotic lesions might have occurred in the lungs of our disease group, while the positive staining was significantly reduced in the CD90⁺hAECs treatment group. The results showed that the CD90⁺hAECs treatment group was significantly better than the unscreened hAECs treatment group in masson staining indicative of fibrosis (FIG. 10).

### Example 10 Immunohistochemical Staining

The paraffin slices were placed at 65° C. for 1 h for deparaffinization.

The slices were dewaxed in xylene solution three times and soaked for 10 min, 10 min, and 5 min sequentially.

The slices were taken out and hydrated in 100% ethanol for 5 min, 100% ethanol for 5 min and 95% ethanol for 2 min. The slices were then rinsed under running water for 5 min.

The slices were placed in antigen repair solution preheated to 98° C. and incubated at a constant temperature of 98° C. for 30 min.

The slices were cooled to room temperature and washed with PBS at room temperature for 5 min.

Added 3% hydrogen peroxide dropwise to the slices and incubated for 10 minutes.

The slices were washed 3 times with PBS for 2 min each time;
Shook the slices dry, added primary antibody dropwise and incubated overnight at 4° C. The slices were washed 3 times with PBS for 2 min each time;
Added Reagent 1 dropwise, incubated at room temperature for 20 min, the slices were washed 3 times with PBS for 2 min each time;
Added Reagent 2 dropwise, incubated at room temperature for 20 min, the slices were washed 3 times with PBS for 2 min each time;
Added DAB dropwise to develop color for 5-20 minutes

The slices were rinsed in running water for 5 min, re-stained with hematoxylin, dehydrated with ethanol gradient, permeabilized with xylene, and sealed with a neutral resin and the examined under microscope.

### Example 11 MACS Sorting

PBMC were resuspended in culture solution to 10\hat{ }7 cells per 100 ul and then packed into sterile tubes, added 10 ul antibody per 100 ul, incubated on ice for 15 min.

Mixed well with resuspend beads, added 10 ul of beads per 100 ul and incubated on ice for 15 min.

Added 3 ml of MojoSort™ Buffer and mixed with cells.

The cells were placed in magnet adsorbing for 5 min.

Pour the liquid out into a collection tube and mixed with the same volume of culture solution, centrifuged at 350 g for 5 min (Repeated steps 3-5 if necessary to improve recovery rate).

Discarded the liquid, the cells were resuspended with complete medium, then cultured or froze.

### Example 12 CFSE Detection of Proliferation

Resuspended 10\hat{ }6-5×10\hat{ }6 cells in 1 ml CFDA SE Labeling Solution.

CFDA SE Stock Solution (1000×) was diluted to 2× with CFDA SE Labeling Solution.

Added 1 ml of CFDA SE stock solution (2×) to step 1, mixed gently and incubated at 37° C. for 10 min.

Added complete medium (with serum) to terminate the reaction, mixed upside down several times.

Centrifuged at 300 g for 5 min and discarded the supernatant.

The precipitate was resuspended by adding 10 ml of complete medium.

Centrifuged at 300 g for 5 min and discarded the supernatant.

The cells were resuspended by adding 10 ml of complete medium and incubated at 37° C. for 5 min in order to promote the residence of CFDA SE within the cells and the entry of unreacted CFDA SE into the complete cell culture medium.

Centrifuged at 300 g for 5 min and discarded the supernatant.

The labeled cells were cultured as needed.

The cells were harvested and the average intracellular fluorescence intensity was measured by FACS.

### Example 13 Lentiviral Infection and Localization of CD90⁺hAECs in Target Organs

Virus-associated component plasmids and vector plasmids were amplified by DH5α and plasmid extraction was performed using endotoxin free plasmid extraction kit.

293T was inoculated into a 10 cm dish and growed to about 80%.

Mixed each plasmid of the virus well, then mixed with water and calcium transfer reagent, and incubated at room temperature for 2 min.

Shook the above solutions and mixed well.

Incubated overnight at 37° C., changed the solution and collected the virus at the appropriate time.

The virus was mixed with appropriate amount of polybrene and added to the target cells to infect them for 48-72 h, and the expression of labeled proteins was detected to determine the infection efficiency.

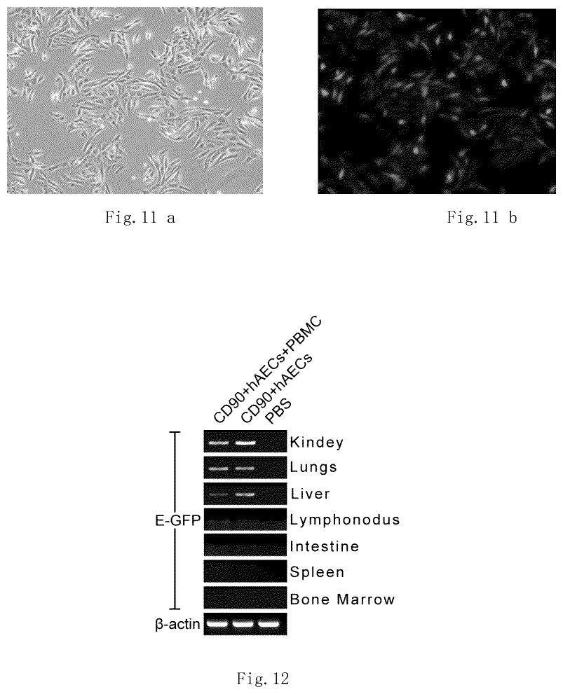

Virus-infected CD90+hAECs were morphologically normal, and green fluorescence micrographs indicated high infection efficiency (FIG. 11). The GFP-tagged CD90⁺hAECs were implanted into NCG mice alone or together with PBMC. One week later, the organs of each mouse were taken, and the mRNA was extracted and reverse-transcribed into cDNA, and the expression of GFP in the tissues was detected by RT-PCR, which was used as an indication of the localization of the CD90⁺hAECs in the tissues. The results showed that GFP was highly expressed in kidney, lung, and liver, but very low in small intestine, spleen, and lymph node, and almost no expression was found in bone marrow. The results showed that CD90⁺hAECs were mainly localized in kidney, lung, and liver (FIG. 12).

The present invention investigated the potential ability of CD90⁺ human amniotic epithelial cells (hAECs) in the treatment of graft-versus-host disease and explores its therapeutic mechanism. The results showed that the CD90⁺ hAECs and PBMC co-transplanted treatment group and the unscreened hAECs treatment group had a better inhibitory effect on aGVHD, and significantly improved the clinical and pathological phenotypes of the mice compared with the disease group, as well as significantly improved the survival rate of the mice.

1. A method of treating and/or improving graft-versus-host disease in a subject in need thereof, comprising administering to the subject an effective dose of human amniotic epithelial cells or cell preparation thereof, wherein the human amniotic epithelial cells (hAECs) are CD90⁺ hAECs cell subset.

2. The method according to claim 1, characterized in that: an effective dose of CD90⁺ human amniotic epithelial cells or cell preparation thereof is administered alone or in combination with other drugs to treat and/or improve graft-versus-host disease, wherein the drugs are selected from the group consisting of methylprednisolone, cyclosporine, tacrolimus mecophenolate mofetil, methotrexate, glucocorticosteroids, azathioprine thalidomide, an anti-T-cell monoclonal antibody (anti-CD3 monoclonal antibody), an antibody against the interleukin-2 receptor, and combinations thereof.

3. The method according to claim 1, characterized in that: the subject is a mammal which is a cow, horse, sheep, monkey, dog, rat, mouse, rabbit or human being.

4-5. (canceled)

6. The method according to claim 1, characterized in that: said cell preparation comprises CD90⁺ human amniotic membrane epithelial cells and a pharmaceutically acceptable carrier.

7. The method according to claim 6, characterized in that: said carrier comprises a physiological buffer selected from the group consisting of physiological saline, phosphate buffer, artificial cerebrospinal fluid, whole serum, and umbilical cord serum.

8. The method according to claim 1, characterized in that: any suitable method can be used to administer CD90⁺ amniotic epithelial cells to the subject, and the method is intravenous injection or intraspinal injection.

9. The method according to claim 8, characterized in that: the administration of CD90⁺ amniotic epithelial cells is repeated or continuous, and the repeated administration methods is administered separately at intervals of at least 7-10 days.

10. The method according to claim 8, characterized in that: CD90⁺ amniotic membrane epithelial cells are planted in a bioabsorbable material, and the bioabsorbable material seeded with cells is surgically implanted into the desired site.

11. The method according to claim 1, characterized in that: the effective dose of CD90⁺ amniotic epithelial cells is changed according to the patient's age, gender, weight, health status and other factors.

12. The method according to claim 11, characterized in that: the dose range of CD90⁺ amniotic epithelial cells administered per time is 10³-10⁹ cells.

13. The method according to claim 12, characterized in that: the dose range of CD90⁺ amniotic epithelial cells administered per time is 10⁶-10⁸ cells.

14. (canceled)

15. A method for isolating CD90+ amniotic membrane epithelial cells from amniotic membrane tissue, comprising the following steps:
(1) Obtaining amniotic membrane from placental tissue through mechanical separation;
(2) The amniotic membrane is washed and then digested with digestive enzymes, and the digested liquid is centrifuged to obtain human amniotic membrane epithelial cells;
(3) Sorting out CD90⁺ human amniotic epithelial cells (hAECs).

16. The method according to claim 15, characterized in that: said method step (1) comprises separating the amniotic membrane from an isolated human placenta, washing it with physiological buffer to remove blood cells, and mechanically removing residual chorion and blood vessels.

17. The method according to claim 16, characterized in that: the human amniotic membrane is obtained with the authorization and consent of the puerpera, the placenta tissue of a healthy puerpera after caesarean section is taken and the entire amniotic membrane is obtained through mechanical separation.

18. The method according to claim 15, characterized in that: said method step (2) uses any conventional technology or method to separate single cells from intact human amniotic epithelial tissue, including mechanical force, enzymatic digestion with one or a combination of proteases selected from the group consisting of collagenase, trypsin, lipase, liberase, and pepsin or a combination of mechanical and enzymatic methods.

19. The method according to claim 15, characterized in that: the screening process of CD90 positive human amniotic epithelial cells in step (3) is: hAECs are stained with CD90 primary antibody, then incubated with fluorescence-conjugated secondary antibody, and analyzed by flow cytometry to sort out CD90⁺ human amniotic epithelial cells.

20. The method according to claim 19, characterized in that: said screening process of CD90⁺ human amniotic epithelial cells in step (3) is: hAECs are stained with CD90 primary antibody(1:20), and then incubated with fluorescence-conjugated secondary antibodies; FACS Calibur instrument Flow cytometry analysis is performed to sort out CD90⁺ human amniotic epithelial cells.

21. The method according to claim 15, characterized in that: the CD90⁺ human amniotic epithelial cells obtained in step 3 are continued to be cultured, and the preferred culture conditions are as follows: inoculate the cells in a petri dish at a density of 1×10⁶-1×10⁸ cells/plate, place the cells in a carbon dioxide incubator for cultivation, and then change the culture medium after the CD90⁺ human amniotic epithelial cells adhere to the wall, and the cells are digested and frozen after the cells cover the plate.

22. The method according to claim 21, characterized in that: the active cell population can be further purified or enriched to reduce stray cells and dead cells after the cells are washed or cultured; isolation of cells in suspension can be accomplished by the following techniques: buoyant density sedimentation centrifugation, differential adhesion to and elution from the solid phase, immunomagnetic beads, fluorescent laser cell sorting (FACS), or other techniques.

23. The method according to claim 21, characterized in that: the medium for culturing CD90⁺ human amniotic epithelial cells is DMEM medium or NPBM medium.

24. The method according to claim 23, characterized in that: F-12, FCS or neural survival factor is added to the basal culture medium.

25. The method according to claim 24, characterized in that: bFGF (basic fibroblast growth factor) and/or EGF (epidermal growth factor) is added to the basal medium.

26. The method according to claim 25, characterized in that: bFGF or EGF is added at a concentration of 1 ng/ml to 100 ng/ml.

27. (canceled)
